(19) United States
(12) Patent Application Publication
(10) Pub. No.: US 2023/0119223 A1
(43) Pub. Date: Apr. 20, 2023

(54) PRESSED POWDER COMPOSITION

(71) Applicant: SHISEIDO COMPANY, LTD., Tokyo (JP)

(72) Inventors: Dhaneswar BRIJLALL, East Windsor, NJ (US); Shoji TAJIMA, East Windsor, NJ (US)

(73) Assignee: SHISEIDO COMPANY, LTD., Tokyo (JP)

(21) Appl. No.: 17/624,149

(22) PCT Filed: Jul. 23, 2020
(86) PCT No.: PCT/IB2020/056976

§ 371 Date: Dec. 30, 2021

Related U.S. Application Data
(60) Provisional application No. 62/878,049, filed on Jul. 24, 2019.

(51) Int. Cl. A61K 8/02; A61K 8/92; A61K 8/25; A61K 8/26; A61K 8/73; A61Q 1/12

(52) U.S. Cl. CPC A61K 8/022; A61K 8/922; A61K 8/25; A61K 8/26; A61K 8/732; A61Q 1/12; A61K 2800/34; A61K 2800/43

(57) ABSTRACT

Provided are cosmetic powder compositions which contain at least one butter, such as shea butter, at least one butter treated powder and a high mass content of spherical powder particles.

### CROSS-REFERENCE TO RELATED APPLICATIONS

The present application claims priority under 37 U.S.C. § 371 to International Patent Application No. PCT/IB2020/056976, filed Jul. 23, 2020, which claims priority to and the benefit of U.S. Patent Provisional Application No. 62/878,049, filed on Jul. 24, 2019. The contents of these applications are hereby incorporated by reference in their entireties.

### FIELD

The present disclosure relates in general to cosmetic compositions and more specifically to pressed powder cosmetic compositions and their methods of making and using.

### SUMMARY

One embodiment is a cosmetic pressed powder composition comprising: (a) at least one butter treated powder; (b) at least one spherical powder; and (c) at least one butter.

Another embodiment is a cosmetic pressed powder composition comprising: (a) from about 5% to about 45% of at least one butter treated powder; (b) from about 20% to about 45% of at least one spherical powder; and (c) from about 0.1% to about 10% of at least one butter, wherein all mass percentages are relative to the total mass of the composition.

### DETAILED DESCRIPTION

Unless otherwise specified “a” or “an” means one or more.

As used herein, the term “about” placed before a specific numeric value may mean±20% of the numeric value; ±18% of the numeric value, ±15% of the numeric value; ±12% of the numeric value; ±8% of the numeric value; ±5% of the numeric value; ±3% of the numeric value; ±2% of the numeric value; ±1% of the numeric value or ±0.5% of the numeric value.

Unless otherwise specified, all content information for ingredients of a composition expressed as % refers to % by mass, relative to the total mass of the composition, unless specified otherwise.

Cosmetic powder compositions may contain a certain level of powdery compounds as well as a certain content of hydrophobic binders, such as oils (e.g., silicone, non-silicone oils), waxes, esters and/or butters. Cosmetic powder compositions are disclosed, for example, in FR2929122, US2014010775, US2014093461, USPN8012459, USPN8765157, and US20100297045.

Many current cosmetic powder compositions use talc as a main powdery ingredient, while containing 5%-10% of spherical powders and a silicone based binder. Using talc as the main powdery ingredient may provide good pressing ability as well as a low cost, while the silicone based treatments of the current powder compositions, which may include, for example, polysiloxanes, such as methicone, dimethicone and/or other silicones oils or polymers, may provide for light feel and good texture of the composition.

The upper limit of the spherical powder content in the current cosmetic powder compositions is usually limited to about 10% because higher spherical powder loads usually lead to a failure of a cake stability (as shown, e.g., by drop test) by the powder compositions.

The present inventors developed solid powder compositions (pressed powders), which may be stable, e.g., being able to pass the drop test, while having a high content of spherical particles. Details of the drop test are disclosed in the Example below.

Spherical powders may be advantageous for reducing appearance of fine lines and pores when used in cosmetic powder compositions. Thus, the present powder compositions may be particularly advantageous for reducing and/or minimizing pore sizes in individuals with enlarged natural skin pore sizes, due to a blurring effect provided by the spherical powders.

The present cosmetic powder compositions may be applied to a keratinous surface, such as a skin, of an individual to reduce a dry appearance. In some embodiments, the present composition may be used alone, for example, as a finisher for setting powders. In some embodiments, the present composition may be used as pre-treatment before applying another cosmetic composition, which may be a liquid cosmetic composition or another powder cosmetic composition.

The present cosmetic powder composition may include a high level of spherical powder particles; at least one butter, such as shea butter, cocoa seed butter, mango butter or a combination thereof; and at least one butter treated powder, such as but not limited to a butter treated mica, kaolin, or starch.

The at least one butter, such as shea butter, and the butter of the at least one butter treated powder, such as a butter treated mica, may be considered as binding ingredients of the present cosmetic powder compositions, i.e., ingredients of the present composition with binding properties. Such binding ingredients may provide for a cohesion of all powdery compounds together, especially in a solid type of composition. Although the present invention is not limited by its theory of operation, the inventors hypothesize that the present powder composition may be stable (e.g., when subject to the drop test) because the binding ingredients may penetrate between the powder particle(s) and thereby hold them together.

The at least one butter and the butter of the at least one butter treated powder may be the same or different. For example, in some embodiments, the at least one butter may be shea butter and the butter of the butter treated powder may be cocoa seed butter.

The present powder composition may be silicone free, i.e. the present powder composition may contain no silicone oils at all (volatile and/or non-volatile) as binders. In some embodiments, the at least one butter may be shea butter (Butyrospermum parkii butter). A butter content, such as a shea butter content, in the powder composition may vary from about 0.1% to about 10%; from about 0.2% to about 10%; from about 0.5% to about 8%; from about 0.8% to about 8%; from about 1% to about 8%; from about 2.0% to about 7.5%; from about 2.5% to about 7% or from about 2.62% to about 6.62% or any value or subrange within these ranges.

The at least one butter used in the composition may include, e.g., shea butter, cocoa seed butter, mango seed butter or any combination thereof.

The present powder compositions may be talc free, i.e. they may contain no talc at all.

A butter used to treat butter treated powder particles may be one or more of, e.g., shea butter, cocoa butter or cocoa seed butter, mango seed butter and their mixtures thereof.

Non-limiting examples of a butter treated powder include those in the Butterpowder® line supplied by Blue Sun International. Particularly useful is Butterpowder MCB91-Q, which is Mica (and) Theobroma Cacao (Cocoa) Seed Butter (and) Tocopheryl Acetate (and) Quercetin.

A content of the butter treated powder in the powder composition may be from about 5% up to about 45% or about 10% up to about 45% or from about 15% up to about 45% or any value or subrange within these ranges. For example, the content of the butter treated powder in the powder composition may be about 5%, about 10%, about 15%, about 20%, about 25%, about 30%, about 35%, about 40%, or about 45% and any value or subrange within this range. In certain embodiments, a content of the butter treated powder, in the powder composition may be about 15% or about 43%.

In some embodiments, the powder composition may further comprise an additional optional powder component(s) such as a filler, which may be, for example, an untreated texturing powder. The filler(s) may be of mineral or organic nature and may provide softness, a matt effect and/or uniformity. The filler(s) may have a different variety of shapes and structure; they may be of lamellar, globular, regular, irregular form, fibers or in any intermediate form. The filler(s) may be surface coated or uncoated. In some embodiments, the fillers may be selected from but not limited to mica, starches, kaolin, and bentone. A content of additional optional powder component(s) (e.g., fillers), such as untreated mica, in the powder composition may be from 0% to about 40%, from about 0% to about 38%, from about 5% to about 35%, from about 10% to about 30%, or from about 15% to about 25% or any value or subrange within these ranges.

Preferably, the compact (i.e., solid) powder composition has a high content of spherical powder particles, i.e. particles that have spherical or a substantially spherical shape, meaning that particles have the shape of sphere and they can be hollow or solid. Spherical powder particles used in cosmetics usually have an average size from 0.1 micron to 250 microns, preferably ranging from about 1 micron to about 150 microns, more preferentially ranging from about 10 microns to about 100 microns or from about 10 microns to 20 microns or from about 1 micron to about 10 microns or from about 3 to about 20 microns or any value or subrange within these ranges.

The powder composition may contain spherical powders at the amount which may be from about 20% to about 45% or from about 22% to about 45% or from about 24% to about 45% or from about 20% to about 32% or from about 22% to about 30% or about 30% to about 45%, or from about 32% to about 42% or from about 35% to about 40%. The spherical powder particle component the composition may include more than one type of spherical powder particles. In some embodiments, the spherical powders may be selected from silica powders, including treated and untreated silica particles, [meth]acrylate powders, silicone elastomers, polyamide powders and their mixtures. Powder(s) of the spherical powder particle component are not butter treated powder particles.

In certain embodiments, the spherical powder particle component of the composition may include multiple different types of spherical particles, such as (A) hydrophobically treated silica spherical powder particles (e.g., treated with silicone oils and/or silicone polymers); (B) [meth]acrylate spherical powder particles, (i.e. spherical acrylate or methacrylate particles); (C) polyamide spherical powder particles; and/or D) silicone elastomers spherical powder particles.

Spherical particles (A) may be, for example, spherical silica particles treated with dimethicone or methicone. Examples of spherical particles (A) include Spheron P1500-030 supplied by Presperse, and those known under Sunsil® line from Sunjin.

In some embodiments, the spherical particles (A) may be present from about 3% to about 25% or from about 5% to about 24% or from about 7% to about 23% or from about 17% to about 22%, from about 17.5% to about 21.5% and even from about 18% to about 21% or any values or subranges within these ranges.

Spherical particles (B) may be, for example, spherical acrylate particles or spherical methacrylate particles, such as spherical methyl metacrylate crosspolymer particles. Examples of spherical particles B) include Ganzpearl® GBM-55COS (acrylates crosspolymer); Ganzpearl® GM-0600 (polymethyl methacylate); Ganzpearl® GM-0800S (polymethyl methacylate); Ganzpearl® GM-0600 (Methyl Methacrylate Crosspolymer); Ganzpearl® GMX-0610 (Methyl Methacrylate Crosspolymer); Ganzpearl® GMX-0810 (Methyl Methacrylate Crosspolymer); Ganzpearl® GMP-0810 (Acrylates Crosspolymer), which are all commercially available from, for example, Presperse, Somerset, N.J., USA, as well as Microsphere® M-series spherical particles, such as polymethyl methacrylate spherical particles (e.g. M-100 and M-101) and polymethyl methacrylate crosspolymer spherical particles (e.g. M-305, M-306, M-330, M-310, M-311 and M-201), which are commercially available from, for example, Matsumoto Yushi-Seiyaku Co., Ltd., Japan.

In some embodiments, the spherical partciles (B) may be present in ranges from about 8% to about 13%, preferably from about 8.5% to about 12.5% and even from 9% to about 12% or any values or subranges within these ranges.

Polyamide spherical particles (C) may be, for example, nylon spherical particles, such as nylon-12 spherical particles. Example of polyamide spherical particles (C) include SP-500 and SP-10 Nylon-12 spherical particles available from Toray Plastics; as well as Anyber Nylon-12 spherical particles, Ganzpearl®GPA-550 Nylon-12 particles and Ganzpearl®GPA-700 Nylon-12 particles, which are all commercially available from, for example, Presperse, Somerset, N.J., USA.

In some embodiments, the spherical partciles (C) may be present in ranges from about 6% to about 10%, or from about 6.5% to about 9.5% or from 7% to about 9% or any values or subranges within these ranges.

Silicone elastomer particles (D) may be selected from KSP-300 spherical particles (diphenyl dimethicone/vinyl diphenyl dimethicone/silsesquioxane crosspolymer), KSP-100 spherical particles (vinyl dimethicone/methicone silsesquioxane crosspolymer); KSP-101 spherical particles (vinyl dimethicone/methicone silsesquioxane crosspolymer); KSP-102 spherical particles (vinyl dimethicone/methicone silsesquioxane crosspolymer which are all commercially available from, Shin-Etsu Silicone.

In some embodiments, the spherical particles (D) may be present in ranges from about 0.5% to about 4%, or from about 1% to about 3.5% or from about 1.5% to about 3% or any values or subranges within these ranges.

In some embodiments, the spherical powder particle component of the composition does not contain untreated silica spherical particles.

Yet in some other embodiments, the spherical powder particle component of the composition may contain untreated silica spherical particles, which may include porous and unporous untreated silica particles. Examples of untreated silica spherical particles include, but not limited to, MSS-500W silica particles, which have an average particle diameter of about 12 microns, oil absorption about 119 g/100 g and refractive index of about 1.47, commercially available from Kobo Products; Satinier M5 silica particles, which have an average particle diameter from about 4 microns to about 8 microns and oil absorption of about 60 ml/100 g, commercially available from Presperse Corporation and Kowa Europe GmbH; Spheron P-1500 particles, which are porous, have an oil absorption of about 60 mL/100 g, an average particle size of about 5 microns to about 7 microns, commercially available from Presperse Corporation; Sunsphere L-51S, which are porous, have an average particle size of about 13 microns and oil absorption of about 150 ml/100 g, commercially available from AGC Chemicals Americas, Inc. The spherical powder particle component of the composition may contain a single type or multiple types, such two types, three type, four types etc., of untreated silica spherical particles. Untreated spherical silica particles of different types may differ from each other in one or more physical property, such as a size (diameter), pore size, absorption properties, such as oil absorption properties.

In some embodiments, some or all of untreated spherical silica particles may have oil absorption properties. In some embodiments, untreated silica ranges having oil absorption properties may have oil absorption rate selected from about 20 m¹/100 g to about 500 ml/100 g, from about 30 ml/100 g to about 300 ml/100 g, or from about 40 ml/100 g to about 200 ml/100 g or from about 50 ml/100 g to about 160 ml/100 g or any value or subrange within these ranges.

An amount of untreated spherical particles may vary. In some embodiments, untreated spherical particles of one or more types may be present in an amount ranging from 0% to about 45% or from about 1% to 40% or from about 2% to about 35% or from about 10% or about 35% or from about 3% to about 30% or from about 15% to about 30% or from about 20% or about 30% or any value or subrange within these ranges. For example, in certain embodiments, an amount of untreated spherical particles may be about 4% or about 6% or about 8% or about 10% or about 12% or about 14% or about 16% or about 18% or about 20% or about 22% or about 24% or about 26% or about 28% or about 30%.

In certain embodiments, the spherical powder particle component of the composition may contain or consist of the combination of untreated spherical particles of (a) one or more types and (b) one of more treated spherical silica particles, such as (A) hydrophobicaly treated spherical silica particles, (B) spherical [meth]acrylate particles; (C) polyamide spherical particles; and (D) silicone elastomer particles. An amount of untreated spherical particles may vary from about 1% to 24% or from about 2% to about 22% or from about 4% to about 20% any value or subrange within these ranges, while treated spherical silica particles, may vary from about 3% to about 25% or from about 5% to about 20% or from about 5% to about 15% or from about 5% to about 12% or any value or subrange within these ranges.

Yet in some embodiments, the spherical powder particle component of the composition may consist untreated spherical particles of one or more types and include no treated spherical silica particles.

Yet in certain embodiments, the spherical powder particle component of the composition may contain or consist of the combination of (A) hydrophobicaly treated spherical silica particles; (B) spherical [meth]acrylate particles; (C) polyamide spherical particles; and (D) silicone elastomer particles.

In some embodiments, an A:B:C:D mass ratio, i.e. a mass ratio between mass contents of spherical particles (A), (B), (C) and (D), may be from about 4.3:2:1.5:0.1 to about 5.5:3.3:2.5:1, preferably from about 4.4:2.1:1.6:0.3) to about 5.4:1.3:2.4:0.9 or even from about to 4.5:2.3:1.8:0.4 to about 5.3:3:2.3:0.8.

As per another embodiment, the mass ratio of butter to butter treated powders is from about 0.4:1 to about 1.5:9, from about 0.5:2 to about 1.4:8.8 or even from about 0.5:3 to about 1.3:8.6.

In addition, the cosmetic powder composition may also contain one or more additional optional ingredients, such as waxes, color pigments, esters, softening/conditioning agents, preservatives, solvents, emollients, surfactants and combinations thereof.

In some embodiments, the additional optional ingredients may be present from about 1% to about 15%, from about 2% to about 12% and preferably from about 3% to about 10%.

### Example 1

A number of pressed powder compositions were evaluated using a “drop test”, which is a test performed according to the following protocol:

1. Drop a product, which may be a powder cake in a compact and a secondary package, from a pan at a height of about 12 inches onto a drop surface, which may be, for example, a lab bench, while ensuring that an open face of the pan points toward the drop surface.

2. Remove the compact from the secondary package to review the cake's integrity. If any powder is broken from the cake's surface, remove such powder with a burst of air.

3. Replace the compact into the secondary package and Repeat Step 1 and Step 2

4. Repeat until all of the cake is removed from the pan, recording a number of drops.

If any cake remains in the pan after 10 drops, then a pressed powder of a particular experiment passes the drop test. Therefore, such powder is considered to be stable. If, however, all the cake fell out of the pan before 10 drops, then a pressed powder for that experiment fails the drop test. A powder that fails a drop test is not considered to be stable.

Samples for the drop test were prepared by pressing a bulk powder composition into a pan, inserting the pan into an appropriate size compact; and placing the compact into a secondary package, such as a carton box.

Pressing conditions were as follows: 9-gram fill mass of the bulk powder, 600 psi for 2 seconds.

Bulk powder compositions were prepared as follows:

1. Prepare a binder phase (Phase C) by mixing all ingredients listed for binder C in Table 1, 2 or 3 in a beaker. Heat the beaker to 60° C. and ensuring that all solids are melted and the mixture is uniform.

2. Prepare a powder phase by mixing ingredient of Phases A1, A2 and B (for specific ingredients see Table 1, 2 or 3) in a metal canister. Mix all the ingredient for A1, A2 and B for 5 seconds using a blender; open a container and clean sides of the container. Repeat the mixing for another 5 seconds. As the result, a powder mixture is formed.

3. Add the binder phase to the powder mixture formed of Phases A1, A2 and B and mix for 20 seconds; open the mixing container and clean its sides. Repeat twice.

For evaluation in the drop test, previously prepared bulk powders were pressed into pressed powder cakes using the following procedure:

1. Weight a bulk powder into a pan

2. Place the pan under a press punch; insert a ribbon between the punch's head and the bulk powder.

3. Press the powder bulk under specific pressure conditions. (9-gram fill mass of the bulk powder, 600 psi for 2 seconds)

4. Remove the pan with pressed powder and insert into final compact. Place compact into secondary package (carton box). Perform drop test.

Results of the drop test for various compositions are presented in Tables 1, 2 and 3.

TABLE 1

| Phase | Ingredient/TRADE Name | INCI Name | Base | Exp 01 |
|---|---|---|---|---|
| A1 | SERICITE CS-15 | MICA | 28.13 | 43.13 |
| Base | BUTTERPOWDER MCB91-Q* | MICA 8.THEOBROMA CACAO (COCOA) SEED | 15.00* | 0.00* |
| | | BUTTER & TOCOPHERYL ACETATE & | | |
| | | QUERCETIN | | |
| | AMIHOPELL | LAUROYL LYSINE | 4.00 | 4.00 |
| | ELESTAB CPN | CHLORPHENESIN | 0.20 | 0.20 |
| A2 | SPHERON P1500-030 | SILICA &. METHICONE | 18.00 | 18.00 |
| Spherical | MICROSPHERE M-305 | METHYL METHACRYLATE CROSSPOLYMER | 12.00 | 12.00 |
| Powders | KSP-300 | Diphenyl Dimethicone/Vinyl Diphenyl | 3.00 | 3.00 |
| | | Dimethicone/Silsesquioxane Crosspolymer | | |
| | SP-500 | NYLON-12 | 7.00 | 7.00 |
| B | | | 0.060 | 0.060 |
| Pigments | | | 0.600 | 0.600 |
| | | | 0.012 | 0.012 |
| C | SHEA BUTTER | BUTYROSPERMUM PARKII (SHEA) BUTTER | 4.62 | 4.62 |
| Binder | CERAPHYL368 | ETHYLHEXYL PALMITATE | 4.62 | 4.62 |
| | PLUROL DIISOSTEARIQUE CG #5793 | POLYGLYCERYL-3 DIISOSTEARATE | 2.76 | 2.76 |
| | | Drop Test Results | Pass - 10x | Fail - 7x |

*the total mass of the composition of the entire raw material.

The data in Table 1 provides evidence that removal of butter coated mica reduces stability of the pressed powder as the cake of Experiment 01, which did not contain butter coated mica, as it failed the drop test (i.e., no powder was left in the compact after 7 drops).

TABLE 2

| Phase | TRADE Name | INCI Name | Base | Exp 01 | Exp 02 | Exp 03 |
|---|---|---|---|---|---|---|
| A1 Base | SERICITE CS-15 | MICA | 28.13 | 28.13 | 38.13 | 28.13 |
| | BUTTERPOWDER | MICA & THEOBROMA CACAO (COCOA) | 15.00* | 15.00* | 15.00* | 15.00* |
| | MCB91-Q | SEED BUTTER & TOCOPHERYL ACETATE & | | | | |
| | | QUERCETIN | | | | |
| | AMIHOPELL | LAUROYL LYSINE | 4.00 | 4.00 | 4.00 | 4.00 |
| | ELESTAB CPN | CHLORPHENESIN | 0.20 | 0.20 | 0.20 | 0.20 |
| A2 | SPHERON P1500-030 | SILICA & METHICONE | 18.00 | 18.00 | 18.0 | 18.0 |
| Spherical | MICROSPHERE M-305 | METHYLMETHACRYLATE | 12.00 | 12.00 | 12.0 | 12.0 |
| Powders | | CROSSPOLYMER | | | | |
| | SUNSPHERE L-51S | SILICA | 0.00 | 4.00 | 0.0 | 0.0 |
| | KSP-300 | Diphenyl Dlmethlcone/Vlnyl Diphenyl | 3.00 | 6.00 | 0.0 | 5.0 |
| | | Dimethicone/Silsesquioxane | | | | |
| | | Crosspolymer | | | | |
| | SP-500 | NYLON-12 | 7.00 | 0.00 | 0.0 | 5.0 |
| B | | | 0.060 | 0.060 | 0.060 | 0.060 |
| Pigments | | | 0.600 | 0.600 | 0.600 | 0.600 |
| | | | 0.012 | 0.012 | 0.012 | 0.012 |
| C | SHEA BUTTER | BUTYROSPERMUM PARKII (SHEA) | 4.62 | 4.62 | 4.62 | 4.62 |
| Binder | | BUTTER | | | | |
| | CERAPHYL 368 | ETHYLHEXYL PALMITATE | 4.62 | 4.62 | 4.62 | 4.62 |
| | PLUROL | POLYGLYCERYL-3 DI ISOSTEARATE | 2.76 | 2.76 | 2.76 | 2.76 |
| | DIISOSTEARIQUE CG | | | | | |
| | #5793 | | | | | |

| | | | | |
|---|---|---|---|---|
| Total Spherical Powders (%) | 40.0 | 40.0 | 30.0 | 40.0 |
| Drop Test Results | Pass - IOx | Fail - 7x | Fall - 7x | Fail - 9x |

*the total mass of the composition of the entire raw material.

The experiments presented in Table 2 demonstrate an effect of spherical powders on an overall cake stability, measured via the drop test. In particular, the base composition having a spherical powder concentration ratio for (silica and methicone):(methyl methacrylate crosspolymer):(Nylon):(diphenyl dimethicone/vinyl diphenyl dimethicone/silsesquioxane) of about 6:4:2.3:1, while containing both shea butter and butter coated mica passed the cake stability test/drop test (i.e., powder remained in the compact after 10 drops), while the compositions of Experiments 01, 02 and 03, which did not have the same spherical powder concentration as the base composition (e.g., one or more of the spherical powders were missing), did not pass the cake stability test.

TABLE 3

| | “Ingredient/ | | Exp 01 | Exp | Exp | Exp | Exp | Exp | Exp | Exp |
|---|---|---|---|---|---|---|---|---|---|---|
| Phase | TRADE Name” | INCI Name | Base | 02 | 03 | 04 | 05 | 06 | 07 | 08 |
| A Base | SERICITE CS-15 | MICA | 28.128 | 28.128 | 38.128 | 28.128 | 28.128 | 28.128 | 28.128 | 28.128 |
| | BUTTERPOWDER | MICA & | 15.00* | 15.00* | 15.00* | 15.00* | 15.00* | 15.00* | 15.00* | 15.00* |
| | MCB91-Q | THEOBROMA | | | | | | | | |
| | | CACAO | | | | | | | | |
| | | (COCOA) SEED | | | | | | | | |
| | | BUTTER & | | | | | | | | |
| | | TOCOPHERYL | | | | | | | | |
| | | ACETATE & | | | | | | | | |
| | | QUERCETIN | | | | | | | | |
| | AMIHOPE | LAUROYL | 4.00 | 4.00 | 4.00 | 4.00 | 4.00 | 4.00 | 4.00 | 4.00 |
| | LL | LYSINE | | | | | | | | |
| | ELESTAB CPN | CHLORPHENESIN | 0.20 | 0.20 | 0.20 | 0.20 | 0.20 | 0.20 | 0.20 | 0.20 |
| A2 | SPHERON | SILICA & | 18.00 | 18.00 | 18.00 | 18.00 | 15.00 | 17.00 | 19.00 | 21.00 |
| Spherical | P1500-030 | METHICONE | | | | | | | | |
| Powders | MICROSPHERE | METHYL | 12.00 | 12.00 | 12.00 | 12.00 | 15.00 | 13.00 | 11.00 | 9.00 |
| | M-305 | METHACRYLATE | | | | | | | | |
| | | CROSSPOLYMER | | | | | | | | |
| | SUNSPHERE | SILICA | 0.00 | 4.00 | 0.00 | 0.00 | 0.00 | 0.00 | 0.00 | 0.00 |
| | L-51S | | | | | | | | | |
| | KSP-300 | Diphenyl | 3.00 | 6.00 | 0.00 | 5.00 | 3.00 | 3.00 | 3.00 | 3.00 |
| | | Dimethicone/Vinyl | | | | | | | | |
| | | Diphenyl | | | | | | | | |
| | | Dimethicone/ | | | | | | | | |
| | | Silsesquioxane | | | | | | | | |
| | | Crosspolymer | | | | | | | | |
| | SP-500 | NYLON-12 | 7.00 | 0.00 | 0.00 | 5.00 | 7.00 | 7.00 | 7.00 | 7.00 |
| B | | | 0.060 | 0.060 | 0.060 | 0.060 | 0.060 | 0.060 | 0.060 | 0.060 |
| Pigments | | | 0.600 | 0.600 | 0.600 | 0.600 | 0.600 | 0.600 | 0.600 | 0.600 |
| | | | 0.012 | 0.012 | 0.012 | 0.012 | 0.012 | 0.012 | 0.012 | 0.012 |
| “C | SHEA | BUTYROSPERMUM | 4.62 | 4.62 | 4.62 | 4.62 | 4.62 | 4.62 | 4.62 | 4.62 |
| Binder” | BUTTER | PARKII (SHEA) | | | | | | | | |
| | | BUTTER | | | | | | | | |
| | CERAPHYL | ETHYLHEXYL | 4.62 | 4.62 | 4.62 | 4.62 | 4.62 | 4.62 | 4.62 | 4.62 |
| | 368 | PALMITATE | | | | | | | | |
| | PLUROL | POLYGLYCERYL-3 | 2.76 | 2.76 | 2.76 | 2.76 | 2.76 | 2.76 | 2.76 | 2.76 |
| | DIISOSTEARIQUE | DIISOSTEARATE | | | | | | | | |
| | CG #5793 | | | | | | | | | |

| | | | | | | | | |
|---|---|---|---|---|---|---|---|---|
| Drop Test Results | 10 | 7 | 9 | 9 | 8 | 12 | 11 | 10 |
| Total concentration | 40.00 | 40.00 | 30.00 | 40.00 | 40.00 | 40.00 | 40.00 | 40.00 |
| of spherical powders | | | | | | | | |
| Formula Notes | Base | Remove | Remove | Increase | De- | De- | Increase | Increase |
| | | Nylon- | Nylon- | KSP- | crease | crease | silica, | silica, |
| | | 12, | 12, KSP- | 300, | silica, | silica, | de- | de- |
| | | replace | 300; QS | de- | increase | increase | crease | crease |
| | | with | with | crease | MMCP | MMCP | MMCP | MMCP |
| | | Silica, | Mica | Nylon- | | | | |
| | | increase | | 12 | | | | |
| | | silicone | | | | | | |
| | | powder | | | | | | |

| | “Ingredient/ | | Exp | Exp | Exp | Exp | Exp | Exp | Exp | Exp |
|---|---|---|---|---|---|---|---|---|---|---|
| Phase | TRADE Name” | INCI Name | 09 | 10 | 11 | 12 | 13 | 14 | 15 | 16 |
| A Base | SERICITE CS-15 | MICA | 28.128 | 28.128 | 28.128 | 43.128 | 0.000 | 28.128 | 28.128 | 28.128 |
| | BUTTERPOWDER | MICA & | 15.00* | 15.00* | 15.00* | 0.00* | 43.13* | 15.00* | 15.00* | 15.00* |
| | MCB91-Q | THEOBROMA | | | | | | | | |
| | | CACAO | | | | | | | | |
| | | (COCOA) SEED | | | | | | | | |
| | | BUTTER & | | | | | | | | |
| | | TOCOPHERYL | | | | | | | | |
| | | ACETATE & | | | | | | | | |
| | | QUERCETIN | | | | | | | | |
| | AMIHOPE | LAUROYL | 4.00 | 4.00 | 4.00 | 4.00 | 4.00 | 4.00 | 4.00 | 4.00 |
| | LL | LYSINE | | | | | | | | |
| | ELESTAB CPN | CHLORPHENESIN | 0.20 | 0.20 | 0.20 | 0.20 | 0.20 | 0.20 | 0.20 | 0.20 |
| A2 | SPHERON | SILICA & | 18.00 | 18.00 | 18.00 | 18.00 | 18.00 | 18.00 | 18.00 | 18.00 |
| Spherical | P1500-030 | METHICONE | | | | | | | | |
| Powders | MICROSPHERE | METHYL | 12.00 | 12.00 | 12.00 | 12.00 | 12.00 | 12.00 | 12.00 | 12.00 |
| | M-305 | METHACRYLATE | | | | | | | | |
| | | CROSSPOLYMER | | | | | | | | |
| | SUNSPHERE | SILICA | 0.00 | 0.00 | 0.00 | 0.00 | 0.00 | 0.00 | 0.00 | 0.00 |
| | L-51S | | | | | | | | | |
| | KSP-300 | Diphenyl | 1.00 | 2.50 | 3.50 | 3.00 | 3.00 | 3.00 | 3.00 | 3.00 |
| | | Dimethicone/Vinyl | | | | | | | | |
| | | Diphenyl | | | | | | | | |
| | | Dimethicone/ | | | | | | | | |
| | | Silsesquioxane | | | | | | | | |
| | | Crosspolymer | | | | | | | | |
| | SP-500 | NYLON-12 | 9.00 | 7.50 | 6.50 | 7.00 | 7.00 | 7.00 | 7.00 | 7.00 |
| B | | | 0.060 | 0.060 | 0.060 | 0.060 | 0.060 | 0.060 | 0.060 | 0.060 |
| Pigments | | | 0.600 | 0.600 | 0.600 | 0.600 | 0.600 | 0.600 | 0.600 | 0.600 |
| | | | 0.012 | 0.012 | 0.012 | 0.012 | 0.012 | 0.012 | 0.012 | 0.012 |
| “C | SHEA | BUTYROSPERMUM | 4.62 | 4.62 | 4.62 | 4.62 | 4.62 | 0.00 | 2.62 | 6.62 |
| Binder” | BUTTER | PARKII (SHEA) | | | | | | | | |
| | | BUTTER | | | | | | | | |
| | CERAPHYL | ETHYLHEXYL | 4.62 | 4.62 | 4.62 | 4.62 | 4.62 | 4.62 | 4.62 | 4.62 |
| | 368 | PALMITATE | | | | | | | | |
| | PLUROL | POLYGLYCERYL-3 | 2.76 | 2.76 | 2.76 | 2.76 | 2.76 | 2.76 | 2.76 | 2.76 |
| | DIISOSTEARIQUE | DIISOSTEARATE | | | | | | | | |
| | CG #5793 | | | | | | | | | |

| | | | | | | | | |
|---|---|---|---|---|---|---|---|---|
| Drop Test Results | 15 | 11 | 11 | 8 | 15 | 7 | 10 | 13 |
| Total concentration | 40.00 | 40.00 | 40.00 | 40.00 | 40.00 | 40.00 | 40.00 | 40.00 |
| of spherical powders | | | | | | | | |
| Formula Notes | De- | De- | Increase | Remove | Remove | Remove | De- | Increase |
| | crease | crease | KSP- | Butter- | Mica, | Shea | crease | Shea |
| | KSP- | KSP- | 300, | powder, | QS with | Butter, | Shea | Butter |
| | 300, | 300, | de- | QS with | Butte- | no QS | Butter by | by |
| | increase | increase | crease | Mica | powder | | 50%, no | 50%, |
| | Nylon- | Nylon- | Nylon- | | | | QS | no QS |
| | 12 | 12 | 12 | | | | | |

*the total mass of the composition of the entire raw material.
Drop test: 10X passed, less than 10-failed

Table 3 provides additional evidence regarding an effect of a particular pressed powder compositions on an overall cake stability, measured via the drop test. The composition of Table 3's base/experiment 01 composition corresponds to the base composition of Tables 1 and 2. Table 3's experiment 02 provides evidence that removal of the nylon-12 particles, while adding 4% of silica particles and increase in KSP-300 particles to 6% leads to a failure in the drop test. Table 3's experiment 03 provides evidence that removal of Nylon-12 and KSP-300 particles while increasing mica's content to 38.128% leads to a failure of the drop test. Table 3's experiment 04 provides evidence that increase of KSP-300 content to 5% while decreasing Nylon-12 content down to 5% leads to a failure of the drop test. Table 3's experiment 05 provides evidence that decrease the silicon/methicone particles content down to 15%, while increasing the methyl methacrylate crosspolymer particles content up to 15% leads to a failure of the drop test. Table 3's experiments 06-08 provide evidence that pressed powder compositions pass the drop test when the silica/methicone particles content varies between 17% and 21%, while the methyl methacrylate crosspolymer particles content varies between 9% and 13% so that a sum of the silica/methicone particles content and the methyl methacrylate crosspolymer particles content remains the same. Table 3's experiments 09-11 provides evidence that pressed powder compositions pass the drop test when the KSP-300 content varies between 1% and 3.5% and the Nylon-12 particles content varies between 6.5% and 9.00% so that a sum of the Nylon-12 particles content and the KSP-300 content remains the same. Table 3's experiment 12 provides evidence that a pressed powder composition without a buttered powder fails the drop test. Table 3's experiment 13 provides evidence that a pressed powder composition with a buttered powder but without unbuttered powder passes the drop test. Table 3's experiment 14 provides evidence that a removal of shea butter leads to a failure of the drop test. Table 3's experiments 15-16 provide evidence that pressed powder compositions pass the drop test when the shea butter content varies from 2.62% to 6.62%.

### Example 2

The following formulation was prepared as disclosed in Example 1:

TABLE 4

| Phase | Trade Name | INCI Name | % |
|---|---|---|---|
| A1 (Base) | | | |
| | SERICITE CS-15 | MICA | 21.577500 |
| | BUTTERPOWDER | MICA, THEOBROMA CACAO (COCOA) | 35.000000 |
| | MCB91-Q | SEED BUTTER, TOCOPHERYL ACETATE, | |
| | | QUERCETIN | |
| | MAGNESIUM | MAGNESIUM MYRISTATE | 4.000000 |
| | MYRISTATE | | |
| | ELESTAB ® CPN | CHLORPHENESIN | 0.200000 |
| A2 (Spherical Powders) | | | |
| | SPHERON | SILICA | 8.000000 |
| | PRESPERSE P-1500 | | |
| | MSS-500W | SILICA | 8.000000 |
| | SATINIER M5 | SILICA | 5.000000 |
| | SUNSPHERE L-51S | SILICA | 5.000000 |
| B (Pigments) | | | |
| | UNIPURE YELLOW LC | IRON OXIDES, LAUROYL LYSINE | 0.134000 |
| | 182 LL | | |
| | UNIPURE RED LC | IRON OXIDES, LAUROYL LYSINE | 0.038400 |
| | 381 LL | | |
| | (PULVERIZED) | | |
| | UNIPURE BLACK LC | IRON OXIDES, LAUROYL LYSINE | 0.000100 |
| | 989 LL | | |
| | (PULVERIZED) | | |
| C (Binder) | | | |
| | LIPEX ® SHEA | BUTYROSPERMUM PARKII (SHEA) | 1.000000 |
| | | BUTTER | |
| | COSMOL 222 | DIISOSTEARYL MALATE | 8.000000 |
| | (RM-00261) | | |
| | O.D.O | CAPRYLIC/CAPRIC TRIGLYCERIDE | 4.000000 |
| | VITAMIN E- | TOCOPHERYL ACETATE | 0.050000 |
| | ACETATE CARE | | |
| | 30499500 | | |
| | | | 100.000000 |

Although the foregoing refers to particular preferred embodiments, it will be understood that the present invention is not so limited. It will occur to those of ordinary skill in the art that various modifications may be made to the disclosed embodiments and that such modifications are intended to be within the scope of the present invention.

All of the publications, patent applications and patents cited in this specification are incorporated herein by reference in their entirety.

1. A cosmetic pressed powder composition comprising:
(a) at least one butter treated powder;
(b) at least one spherical powder; and
(c) at least one butter, wherein the butter treated powder is selected from powders treated with cocoa butter, mango butter, shea butter and mixtures thereof.

2. The cosmetic pressed powder composition of claim 1 comprising:
(a) from about 5% to about 45% of the at least one butter treated powder;
(b) from about 20% to about 45% of the at least one spherical powder; and
(c) from about 0.1% to about 10% of the at least one butter, wherein all mass percentages are relative to the total mass of the composition.

3. The composition according to claim 2, wherein the at least one butter treated powder is selected from a butter treated mica, a butter treated starch, a butter treated kaolin and a mixture thereof.

4. The cosmetic powder composition of claim 2, wherein the at least one butter treated powder comprises a butter treated mica powder.

5. (canceled)

6. The composition according to claim 4, wherein the butter treated mica powder is a mica powder treated with a cocoa seed butter.

7. The composition according to claim 2, wherein the at least one spherical powder comprises at least one spherical powder selected from: (A) hydrophobically treated silica spherical particles, (B) (meth)acrylate spherical particles, (C) polyamide spherical particles, (D) silicone elastomer spherical particles, and (E) untreated silica particles and mixtures thereof.

8. The composition according to claim 2, wherein the at least one spherical powder is a mixture of A) hydrophobicaly treated silica spherical particles, B) (meth)acrylate spherical particles, C) polyamide spherical particles and D) silicone elastomer spherical particles.

9. The composition according to claim 8, wherein the at least one spherical powder is selected from methicone treated silica spherical particles, methyl methacrylate crosspolymer spherical particles, nylon-12 spherical particles, diphenyl dimethicone/vinyl diphenyl dimethicone/silsesquioxane crosspolymer spherical particles and mixtures thereof.

10. The composition according to claim 8, wherein a mass ratio between mass contents of spherical particles (A), (B), (C) and (D) is from about 4.3:2:1.5:0.1 to about 5.5:3.3:2.5:1, wherein all mass ratios are relative to the total mass of the composition.

11. The composition of claim 2, wherein the at least spherical powder comprises untreated silica spherical particles.

12. The composition of claim 11, wherein the at least spherical powder consists of untreated silica spherical particles.

13. The composition according to claim 2, wherein the at least one butter is selected from cocoa butter, mango butter, shea butter and mixtures thereof.

14. The composition according to claim 2, wherein the at least one butter is shea butter.

15. The composition according to claim 2, wherein the mass ratio of the at least one butter to the at least one butter treated powder is from about 0.4:1 to about 1.5:9.

16. The composition according to claim 2, wherein the composition is silicone free.

17. The composition according to claim 2, wherein the composition is talc free.

18. A cosmetic method comprising applying the cosmetic powder composition of claim 1 to a human skin.

19. A cosmetic pressed powder composition comprising:
(a) at least one butter treated powder;
(b) at least one spherical powder; and
(c) at least one butter, the at least one butter is selected from cocoa butter, mango butter, shea butter and mixtures thereof.

20. The cosmetic composition of claim 19 comprising:
(a) from about 5% to about 45% of the at least one butter treated powder;
(b) from about 20% to about 45% of the at least one spherical powder; and
(c) from about 0.1% to about 10% of the at least one butter, wherein all mass percentages are relative to the total mass of the composition.

21. A cosmetic method comprising applying the cosmetic powder composition of claim 19.
